(19) United States
(12) Patent Application Publication
(10) Pub. No.: US 2004/0064115 A1
(43) Pub. Date: Apr. 1, 2004

(54) Disposable articles having a failure detection system

(76) Inventors: Tarun K. Arora, Edison, NJ (US); Karen N. Hagerty, Doylestown, PA (US); H. Michael Moscherosch, Doylestown, PA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/261,257

(22) Filed: Sep. 30, 2002

(51) Int. Cl. A61F 13/15; A61F 13/20

(52) U.S. Cl. 604/361

(57) ABSTRACT

A disposable article, e.g., useful as a sanitary protection device. The disposable article has a primary structure and a liquid-sensitive signaling element in liquid communication with the primary structure. Upon exposure to fluid, the liquid-sensitive signaling element expands to signal the user that the article is approaching capacity.

### FIELD OF THE INVENTION

The present invention relates to disposable articles having a liquid-sensitive, signaling element that functions as a failure detection device. The failure detection device is capable of signaling the user to change the article as it is approaching capacity or otherwise near failure.

### BACKGROUND OF THE INVENTION

Disposable articles are often used as sanitary protection articles that are designed to contain a particular amount of body exudates such as menstrual fluid or urine. The amount of menstrual fluid absorbed by a napkin, pantiliner, or interlabial device or urine absorbed by an incontinence article can vary depending on absorbency levels. In order to ascertain whether an article has reached its absorbent capacity, it must be viewed. Often, a user will remove a disposable article before it has reached its absorbent capacity in order to prevent fluid from soiling the user's undergarment when its absorbent capacity is exceeded. Once the absorbent capacity is exceeded, the excess bodily fluids can soil the user's clothing.

Ito et al., U.S. Pat. Nos. 4,357,938 and 4,447,240, disclose a disposable diaper having water-absorbing shrinkable fibers, which shrink in the direction of the fiber length and are rendered elastic on absorption of water, that are fixed to and extend along the central portion in the lengthwise direction of the disposable diaper. When the water-absorbing shrinkable fibers become wetted after the diaper is wetted, the water-absorbing shrinkable fibers shrink to form an elastically gathered side edge of the diaper.

Many external sanitary protection devices currently attach to an undergarment and are made from multiple layers of different materials. For example, a pantiliner may have a cover, an absorbent core, and a barrier. The cover is fluid permeable so that fluid can pass through and be absorbed by the absorbent core. The barrier is typically fluid impermeable so as to prevent fluid from soiling the user's undergarment.

Carlucci et al., U.S. Pat. No. 6,191,340 B1 discloses a disposable absorbent article which is substantially flat prior to use for wearing adjacent a body discharge area, having a body facing surface and a garment facing surface. The absorbent core of the article has a body facing surface and a garment facing surface, and has an expanding layer for expanding the article into a tridimensional structure while being worn by a user. The expanding layer includes a number of smaller expanding elements that are activated by body fluids, are decoupled from one another, and are capable of expanding substantially in only one direction upon activation by body fluids.

However, none of the known references use such absorption-alterable materials in an external pad or external device to signal a user that the article is approaching absorbent capacity. Thus, there is a need for a failure detection system that signals the user to change the disposable absorbent article prior to soiling the user's clothing.

### SUMMARY OF THE INVENTION

It is an object of the present invention to provide a disposable article having a failure detection system that signals the user to change the article prior to its soiling of a user's clothing.

In accordance with one embodiment of the present invention, a disposable article having a primary structure capable of being directed toward a source of bodily fluids during use and an expandable, liquid-sensitive signaling element in liquid communication with the primary structure is disclosed. The signaling element is separated from the source of bodily fluids during use by the primary structure, and it has resilient material held in a strained configuration that is capable of expanding to a relatively unstrained configuration upon exposure of the signaling element to bodily fluids.

In accordance with another embodiment of the invention, a disposable incontinence device including a primary structure capable of intravaginal support of a user's urinary system and a liquid-sensitive signaling element capable of being located adjacent the user's urethra is disclosed. The signaling element has resilient material held in a strained configuration that is capable of reverting to a relatively unstrained configuration upon exposure of the signaling element to saline solution.

In accordance with another embodiment of the invention, an intravaginal disposable article including a primary structure sized for insertion into a user's vagina and a liquid-sensitive signaling element in liquid communication with the primary structure is disclosed. The primary structure has a first end capable of being directed toward a source of bodily fluids during use and a second end, opposite the first. The signaling element is separated from the source of bodily fluids during use by the primary structure, and it has resilient material held in a strained configuration that is capable of reverting to a relatively unstrained configuration upon exposure of the signaling element to bodily fluids.

Other aspects of the invention include methods of making such devices and methods of using such devices in the control of aqueous vaginal fluids.

### BRIEF DESCRIPTION OF THE DRAWING

FIG. 1 shows a plan view of an embodiment of the invention showing a pantiliner in a dry state;

FIG. 2 is an exploded cross-section of the pantiliner of FIG. 1 along line AA;

FIG. 3 is an exploded cross-section of FIG. 1 after fluid absorption and expansion of the signaling element;

FIG. 4 is a plan view of another embodiment of the invention showing the disposable absorbent article as a pantiliner;

FIG. 4A is a plan view of the embodiment of FIG. 4 after fluid absorption and expansion of the signaling element;

FIG. 5 is a plan view of another embodiment of the invention showing the disposable absorbent article as a pantiliner;

FIG. 6 shows an example of a block of signaling element having multiple cuts;

FIG. 7 is a perspective view of an uncompressed signaling element arranged on a barrier layer of a pantiliner;

FIG. 8 shows the signaling element of FIG. 7 as compressed.

### DETAILED DESCRIPTION OF THE PREFERRED EMBODIMENTS

Further characteristics and advantages of the invention will become clear from the following detailed description, appended drawings, and non-limiting examples.

As used herein the specification and the claims, the term “liquid communication” and variants thereof relate to the ability of liquid to flow from one element to another.

As used herein the specification and the claims, the term “to saturate” and variants thereof mean to cause to be thoroughly soaked, imbued, or penetrated and include to provide sufficient liquid to dissolve, weaken, or otherwise render a coating insufficient to constrain a resilient material.

As used herein the specification and the claims, the term “resilient” and variants thereof means the capability of a strained body to recover its size and shape after deformation caused especially by bending, compressing, twisting, stretching or any combination thereof. Resilient material can go from a relaxed configuration to a strained configuration to a relaxed configuration at least once and preferably a number of times without losing substantially the ability to recover its original shape. Resilient material in a strained configuration possesses strain energy or potential energy of deformation. It may be desirable that the resilient material be constrained by a coating, sheath or cover, which would weaken upon exposure to fluid.

The present invention is directed to novel disposable articles that are used in contact with a user's body. The disposable articles have a liquid-sensitive, resilient signaling element that functions as a failure detection device. Examples of such articles include, without limitation, external sanitary protection articles such as sanitary napkins, pantiliners, and interlabial devices; absorbent incontinence articles such as diapers and incontinence pads and guards; wound care articles such as bandages; and the like.

The disposable articles include a primary structure and a liquid-sensitive signaling element. The primary structure will generally absorb the passage of, especially, bodily fluids. Thus, the primary structure will generally comprise an absorbent structure. The liquid-sensitive signaling element is capable of expanding in size upon contact with sufficient liquid in a manner to provide a detectable signal to the user.

External disposable devices are suitable for placement adjacent a mammalian body surface and are substantially planar (i.e., having a thickness less than its width and substantially less than its length). For example, the invention can be used in an external sanitary protection device, such as a sanitary pad (e.g., a sanitary napkin or a pantiliner). In a useful embodiment, the sanitary pad has a thickness of less than about 5 mm.

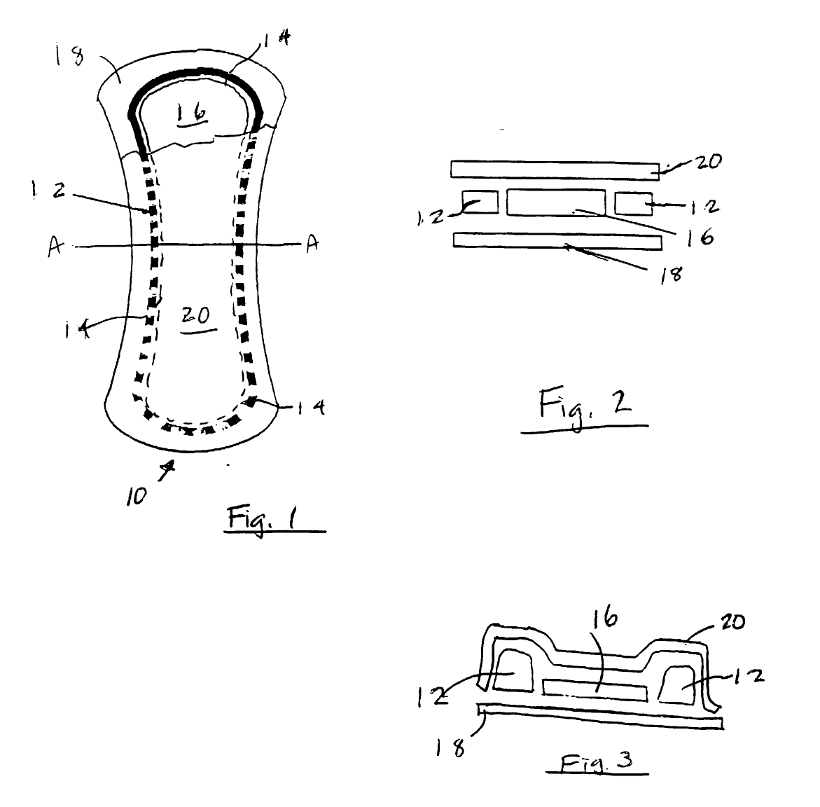

As seen in FIGS. 1-3, the disposable article 10 is a pantiliner having a liquid-sensitive, signaling element 12 located about the periphery 14 of the primary structure 16, in this case an absorbent structure. The pantiliner of FIGS. 1-3 includes an absorbent structure as a primary structure 16, a signaling element 12, a barrier 18 and cover 20. While absorbent structure 16 is shown in FIG. 1 as having a “peanut” shape or having concave longitudinal sides, absorbent structure 16 may have any shape such as rectangular, triangular, etc. While the signaling element 12 generally has the shape of at least part of the periphery 14 of absorbent structure 16, other embodiments of this invention may have other desirable shapes.

When sufficient absorbed fluids reach the periphery 14 of the absorbent structure 16 to be communicated to the signaling element 12, the signaling element 12 can expand rapidly. The expansion of absorbent structure 16 generally occurs over a relatively long period of time as it absorbs the bodily fluids. This is generally a relatively slow expansion and is not usually detectable by the user. In addition, the overall absorbent structure 16 usually expands to a lesser extent than the signaling element 12. Therefore, the expansion of the signaling element 12 is more detectable than that of the absorbent structure 16.

FIG. 2 shows the cross-sectional view of FIG. 1 before use and FIG. 3 shows the cross-sectional view of FIG. 1 after exposure to fluid sufficient to saturate the absorbent structure 16 of the pantiliner. As can been seen in FIG. 3, liquid-sensitive, resilient signaling element 12 has expanded.

FIG. 4 shows another embodiment of the invention where signaling element 12′ is located in the central or crotch portion 11 of the absorbent structure 16′ of a pantiliner 10. In this embodiment, signaling element 12′ does not extend to a first end portion 22 or a second end portion 24. The signaling element 12′ can expand both upward and outward, as shown in FIG. 4A. The signaling element 12′ can also expand outwardly beyond the outer edge of the pantiliner, which may provide improved body fit along the periphery of the product. Such improved fit may act as a gasket to reduce leakage further.

FIG. 5 shows another embodiment of the invention. Signaling element 12″ is made of two strips of resilient material, each of which goes from first end portion 22 to second end portion 24. This embodiment is especially useful for resilient devices in which absorbent structure 16″ is rectangular in shape. This embodiment also shows optional secondary structures 26, in this case they are absorbent, outside of the signaling elements 12″.

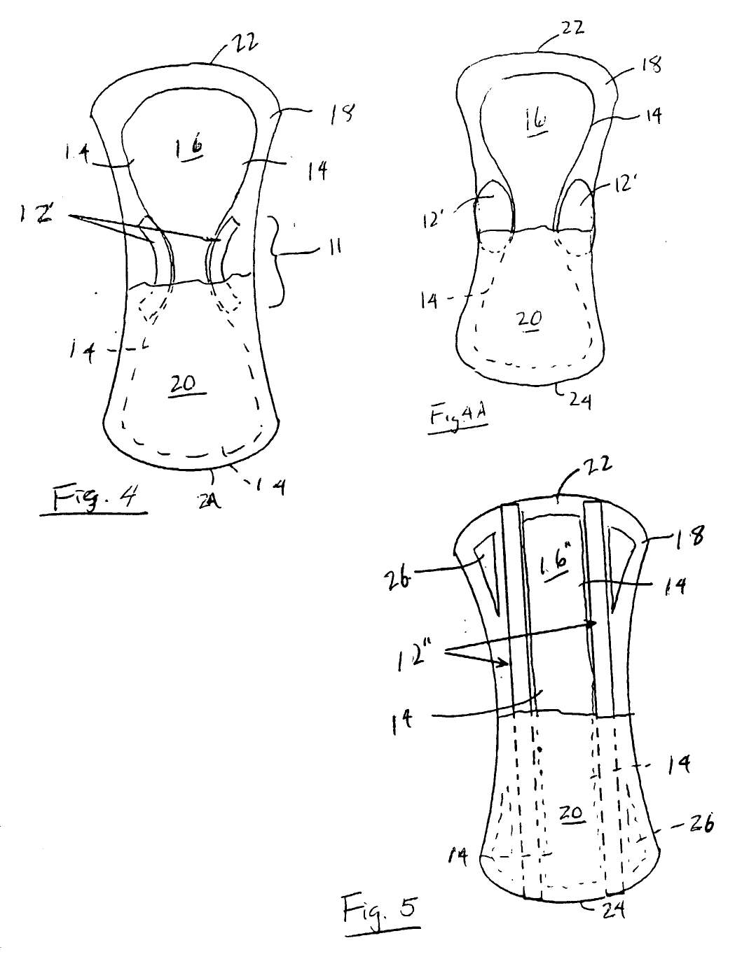

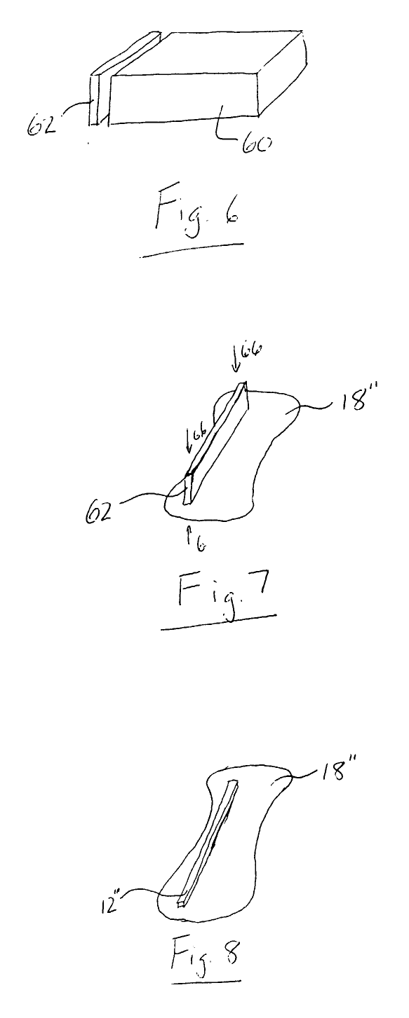

FIGS. 6-8 are illustrative of how the signaling element 12 may be formed. For example, a supply of resilient material in the form of a block 60 may be cut into slices 62 as seen in FIG. 6. For example, the block may have a length that corresponds to that of the periphery of the primary structure with which the signaling element is associated (or at least a portion thereof). The slices may have any desired height, for example, the pre-compression height may be about 25 mm (about 1 inch) or slightly more or significantly less, as short as about 6 mm (about ¼ inch). This slice 62 can be oriented on the barrier 18″ and combined with the primary structure (not shown) to form the disposable article. The slice 62 can then be compressed in the direction of arrows 64 and 66 to form the elongate signaling element 12″ as shown in FIG. 8 having a thickness, e.g., of less than 5 mm. Of course, the resilient material may be compressed at other points in the manufacturing process, and the signaling element may be attached to other elements of the article, such as the cover, an intermediate layer, etc. It is believed that a user will not greatly notice these unexpanded dimensions, however, it is believed that the user would detect the expanded structure. If the primary structure 12 is a compressed structure, the order of compression of this structure 12 and the signaling element 12 may occur in any order.

Signaling element 12 may comprise any absorbent material that is capable of expansion upon absorption of bodily fluids or any resilient material that is capable of being strained and held in the strained configuration in a dry state and that, upon exposure to liquid (such as blood, menstrual fluid, mucus, saline solution, urine, etc.), expands to its original, relaxed configuration. The expansion of signaling element 12 may be primarily radially (i.e., generally uniformly outward from a central axis), longitudinally (i.e., lengthwise), sideways (i.e., generally perpendicular along one line perpendicular to a central axis), or any combinations thereof.

Examples of absorbent materials useful in the present invention include but are not limited to hydrogels; modified starches; gelatin; other water soluble polymers that by proper treatment (including but not limited to crosslinking) can be modified to be insoluble while maintaining is affinity to water such as methyl- and hydroxypropyl-methyl-cellulose and derivatives; hydroxyethylcellulose; carboxymethylcellulose; polyvinyl alcohol, polyvinylpyrrolidone, polyacrylic acid and its homologues (a common class of superabsorbents), polyacrylamide, ethylene oxide polymers and polyethylenimine; and the like.

The strained condition of the resilient material may be achieved by compression in one or more of the following: monoaxially (such as along a longitudinal axis), biaxially (such as along two perpendicular axes), or radially (substantially uniformly inwardly toward a central axis). Essentially, the resilient material may go from a natural, relaxed configuration into a strained configuration and back to the relaxed configuration (when wet). Examples of such resilient materials include but are not limited to spongy materials, such as of cellulose, polyvinyl alcohol, polyvinyl acetal or formal, polyurethane, or other suitable (usually hydrophilic) foam or sponge material (including open cell sponge); compressed cellulosic fibers; compressed springs made from plastic, metal or any shape memory material; and combinations of different materials to impart variations in composition, density, and porosity; and the like. It is helpful if the materials are biocompatible. For example, a cellulose foam material such as cellulosic sponge may be used. In a natural state, the cellulosic material is spongy and has large pores. In a strained configuration, the cellulosic material is stiffer. Upon relaxation, the cellulosic material “springs” into the spongy state.

The signaling element 12 can be formed into any useful shape. Examples, of the strained configuration of the signaling element 12 are represented in FIGS. 1, 4, 5, and 8. Other shapes will be recognized by the skilled artisan, including without limitation, spherical, ovoid, short disks, long cylinders, cubes, diamonds, etc.

Signaling element 12 has an initial volume prior to use. Upon sufficient penetration of fluid, the signaling element 12 expands to an increased volume. In one form of the present invention, the release of the stored potential energy of a compressed resilient material may cause the liquid-sensitive signaling element to expand quickly from the initial volume to a second volume during use. This can be measured in the product by exposing the signaling element to sufficient 0.9 wt-% aqueous saline solution to saturate the signaling element. Preferably, the resilient material in the signaling element changes to its second volume within about 5 seconds, and more preferably within about 2 seconds. The user may then feel this relatively quick movement or motion as a discernible tactile sensation. This sensation would then prompt the user to remove the disposable article and replace it with a new one.

Alternately, it may be the change in volume of the liquid-sensitive signaling element that the user feels. In other words, the signaling element has an initial volume (volume before use or testing) and a second volume achieved after saturation with a 0.9 wt-% aqueous saline solution, and the second volume is larger than the initial volume, generally from about 2 to 20 times (200 to 2000%) its initial volume, and usually from 5 to 15 times (500% to 1500%) its initial volume. The rate at which the liquid-sensitive signaling element expands is not critical in this alternate form of the invention.

The signaling element may be formed of a single unit, or it may have multiple components. Whichever signaling element used, the change in the signaling element should be subtle enough not to startle the user and yet to elicit an appreciable tactile sensation.

In general, it is necessary that the expansion be significant and of enough volume to be felt by the user. In one embodiment, signaling element 12 is formed from a cellulosic material, such as regenerated cellulosic sponge material. In a natural state, the cellulosic material is spongy and has large pores. In a strained configuration, the cellulosic material is stiffer. Upon relaxation, the cellulosic material “springs” into the spongy state.

The signaling element may have a coating, cover or sheath (“cover”), which will advantageously be dimensioned and configured to contain the signaling element. The cover may be dissolvable or expandable to prevent it from significantly impeding the expansion of the signaling element upon exposure to sufficient moisture. It is understood that in some instances differential compression by the cover may beneficially provide areas of the resilient material having different effective densities. The cover may be used to prevent relaxation or pre-mature expansion until sufficiently weakened by an aqueous or a proteinaceous fluid, or it may be distortable to be capable of expansion in multiple directions and move with the signaling element 12.

Additionally, covering or coating the resilient material may be used to isolate the signaling element 12 from the user's body. For example, the cover may prevent any fibers or loose or particulate materials from being left on the body after removal, or it may enclose a spring-like element to protect the user. It has also been found that if the resilient material is, for example, compressed sponge, the resilient material may be stiff and may therefore cause irritation to the vaginal walls until the sponge is saturated. The coating may be any material such as a lubricant, ointment, polymeric film, or the like, which results in a smooth supple outer surface. Useful materials may also be biocompatible. The coating should prevent adherence to body and should ease its removal. Polymeric materials that may be used include, but are not limited to, polyethylene, polypropylene, polyurethane, ethylene vinyl acetate, and silicone; certain polymers can be subjected to a gas plasma or corona discharge treatment, to increase their inherent lubricity. Other materials used may include, without limitation, starch, gelatin, candle wax, polyethylene glycol, polyvinyl alcohol, hydroxypropylcellulose, and polyvinyl propylene, etc. The resilient material may be encased in a capsule formed by a coating or cover.

### EXAMPLE 1

Samples of regenerated cellulosic sponge were cut into approximately 6.5 (L₁) by 6.5 (W₁) by 2.8 (H₁) mm cubes. The samples were individually weighed (w₁) prior to wetting with 0.9 wt-% saline solution. The time required for the saline solution to be absorbed was measured. After wetting, the samples were measured (e.g., L₂) and weighed (w₂). All dimensions were measured in millimeters.

TABLE 1

| Sample | Length | Width | Height | Weight | Absorption |
|---|---|---|---|---|---|
| Number | (L₂ − L₁) | (W₂ − W₁) | (H₂ − H₁) | (w₂ − w₁) | time (sec.) |
| 1 | 0.5 | 0.5 | 22.14 | 1.123 | 2 |
| 2 | 0.5 | 0.5 | 22.26 | 1.002 | 2 |
| 3 | 0.0 | 0.5 | 22.26 | 1.124 | 2 |
| 4 | 0.0 | 0.5 | 22.15 | 1.031 | 2 |
| 5 | 0.5 | 0.5 | 22.2 | 0.997 | 2 |
| Average | 0.3 | 0.5 | 22.2 | 1.055 | 2 |

Each sample absorbed approximately 1 gram of fluid within 2 seconds. Absorption of the fluid resulted in an increase of approximately 800% in height.

The foregoing description is intended as illustrative and is not to be taken as limiting. Still other variations are possible without departing from the spirit and scope of this invention and will readily present themselves to one skilled in the art.

What is claimed is:

1. A disposable article comprising:
a) a substantially planar primary structure having an outer perimeter and suitable for placement adjacent a mammalian body surface; and
b) an expandable, liquid-sensitive signaling element arranged and configured adjacent at least a portion of said outer perimeter, the signaling element comprising resilient material held in a strained configuration that is capable of expanding to a relatively unstrained configuration upon exposure of the signaling element to bodily fluids.

2. The disposable article of claim 1 wherein the primary structure comprises an absorbent structure.

3. An external sanitary pad comprising the disposable article of claim 1.

4. The external sanitary pad of claim 3, wherein the pad has a thickness of less than about 5 mm.

5. The disposable article of claim 1, wherein the signaling element expands to at least about 200% of the initial volume after exposure of the signaling element to a saturating amount of 0.9 wt-% saline solution.

6. The disposable article of claim 5, wherein the signaling element expands to at least about 500% of the initial volume after exposure of the signaling element to a saturating amount of 0.9 wt-% saline solution.

7. The disposable article of claim 1, wherein the signaling element comprises sponge material.

8. The disposable article of claim 7, wherein the sponge material comprises regenerated cellulosic sponge.

9. The disposable article of claim 7, wherein the sponge material comprises foam.

10. The disposable article of claim 1, wherein the signaling element is surrounded with a water soluble material.

11. The disposable article of claim 1, wherein the signaling element comprises absorbent material.

12. The disposable article of claim 11, wherein the absorbent material is compressed.

13. The disposable article of claim 11, wherein the absorbent material is selected from the group consisting of hydrogels, modified starches, gelatin, methyl-cellulose, hydroxypropyl-methyl-cellulose, hydroxyethylcellulose, carboxymethylcellulose, polyvinyl alcohol, polyvinylpyrrolidone, polyacrylic acid, polyacrylamide, ethylene oxide polymers, polyethylenimine, and derivatives and combinations thereof.

14. A method of making a disposable article comprising the steps of:
a) forming a primary structure;
b) manipulating resilient material into a deformed condition;
c) restraining the resilient material in the deformed condition to form a signaling element; and
d) attaching the signaling element to the primary structure.

15. The method of claim 14 wherein the signaling element is attached to the primary structure in the deformed condition.

16. The method of claim 14 wherein the signaling element is attached to the primary structure before the restraining step.
